(19) United States
(12) Patent Application Publication
(10) Pub. No.: US 2005/0202488 A1
(43) Pub. Date: Sep. 15, 2005

(54) Assay for therapies that inhibit expression of the cytosolic Cu/Zn superoxide dismutase (SOD1) gene

(75) Inventors: Robert H. Brown, Needham, MA (US); Wendy Broom, Methuen, MA (US)

Correspondence Address: WOLF GREENFIELD & SACKS, PC;FEDERAL RESERVE PLAZA, 600 ATLANTIC AVENUE, BOSTON, MA, 02210-2211, US

(73) Assignee: The General Hospital Corporation, Boston, MA (US)

(21) Appl. No.: 11/070,868

(22) Filed: Mar. 2, 2005

Related U.S. Application Data
(60) Provisional application No. 60/549,326, filed on Mar. 2, 2004.

(51) Int. Cl. C12Q 1/68; C12Q 1/66

(52) U.S. Cl. 435/6; 435/8

(57) ABSTRACT

Amyotrophic lateral sclerosis (ALS) is a neurodegenerative condition with a prevalence of 2-3 per 100,000 people, involving the loss of large motor neurons in the brain and spinal cord. It is characterized by progressive paralysis that leads to death typically in 3-5 years. Familial ALS accounts for 10% of all ALS cases and approximately 25% of these cases are due to mutations in the Cu/Zn superoxide dismutase gene (SOD1). The invention relates to a high throughput assay and methods for detecting inhibitors. The invention also relates to methods of using the inhibitors to treat and prevent disease.

### RELATED APPLICATIONS

This application claims priority under 35 U.S.C. § 119(e) from U.S. provisional patent application Ser. No. 60/549,326, filed Mar. 2, 2004, the entire disclosure of which is incorporated herein by reference.

### FIELD OF THE INVENTION

The invention relates to a high throughput assay and methods for detecting inhibitors. The invention also relates to methods of using the inhibitors to treat and prevent disease.

### BACKGROUND OF THE INVENTION

Amyotrophic lateral sclerosis (ALS) is a neurodegenerative condition with a prevalence of 2-3 per 100,000 people, involving the loss of large motor neurons in the brain and spinal cord. It is characterized by progressive paralysis that leads to death typically in 3-5 years. Unfortunately, it remains uniformly lethal; there is no drug that significantly slows the progress of paralysis in this disease. Familial ALS accounts for 10% of all ALS cases and approximately 25% of these cases are due to mutations in the Cu/Zn superoxide dismutase gene (SOD1) (Rosen, D. R., (1993), Mutations in Cu/Zn superoxide dismutase gene are associated with familial amyotrophic lateral sclerosis, Nature, 364:362; Andersen, P. M., et al., (2003), Sixteen novel mutations in the Cu/Zn superoxide dismutase gene in amyotrophic lateral sclerosis: a decade of discoveries, defects and disputes, Amyotroph Lateral Scler Other Motor Neuron Disord., 4:62-73). SOD1 is a predominantly cytoplasmic enzyme that catalyzes the breakdown of toxic superoxide ions to oxygen and hydrogen peroxide, which in turn is degraded by glutathione peroxidase or catalase to water. Several lines of evidence argue that the mutant SOD1 protein is neurotoxic through an acquired, adverse function, as yet not explicitly defined.

Particularly convincing is the observation that transgenic expression of high levels of mutant SOD1 protein in mice produces a motor neuron disease phenotype, with age of onset and disease duration dependent on copy number (Gurney, M. E., et al., (1994), Motor neuron degeneration in mice that express a human Cu,Zn superoxide dismutase mutation, Science, 264:1772-5; Ripps, M. E., et al., (1995), Transgenic mice expressing an altered murine superoxide dismutase gene provide an animal model of amyotrophic lateral sclerosis, Proc Natl Acad Sci U.S.A., 92:689-93). In this experimental model transgenic mice lines expressing SOD1-G93A at approximately five times the endogenous level developed a motor neuron disease phenotype, whereas those with a lower copy number did not. At three months of age mice show limb weakness that can be assayed by looking at hind limb splaying when the mice are picked up by the tail. Stride length is also reduced and after a further 14 days paralysis of one of more limbs is noted. SOD1-G93A transgenic mice are moribund at around 5 months. Pathology similar to that seen in ALS patients is observed in the mice. The majority of mutant SOD1 proteins retain catalytic activity; this finding and the genetics of the SOD1 mutations (dominantly inherited) suggest that SOD1-mediated ALS is not a consequence of loss of SOD1 dismutation function, but instead arises from one or more acquired, cytotoxic functions of the mutant SOD1 protein (Rosen, D. R. et al., (1993), Mutations in Cu/Zn superoxide dismutase gene are associated with familial amyotrophic lateral sclerosis, Nature, 364:362). Indeed transgenic mice with three different SOD1 mutations were found to have increased or unchanged SOD1 activity in the brain and spinal cord (Gurney, M. E., et al., (1994), Motor neuron degeneration in mice that express a human Cu,Zn superoxide dismutase mutation, Science, 264:1772-5; Ripps, M. E., et al., (1995), Transgenic mice expressing an altered murine superoxide dismutase gene provide an animal model of amyotrophic lateral sclerosis, Proc Natl Acad Sci U.S.A., 92:689-93). This hypothesis is further supported by the observation that SOD1 knockout animals do not develop an overt motor neuron disease phenotype although their response to oxidative stress is defective (Reaume, A. G., et al., (1996), Motor neurons in Cu/Zn superoxide dismutase-deficient mice develop normally but exhibit enhanced cell death after axonal injury, Nat Genet., 13:43-7). That ALS arises from an acquired property of mutant SOD1 protein is also suggested by the observation that a patient who apparently received two copies of the mutant SOD1 gene (one from each of her parents who were first cousins) developed a profoundly severe form of ALS that developed in her teen years and was lethal in only a few weeks (Hayward, C., et al., (1998). Homozygosity for Asn86Ser mutation in the CuZn-superoxide dismutase gene produces a severe clinical phenotype in a juvenile onset case of familial amyotrophic lateral sclerosis [letter]. J Med Genet., 35(2): p. 174).

To date, Rilutek® (Riluzole) is the first and only drug to receive FDA approval for the treatment of ALS. Unfortunately, its clinical effects are very modest at best. Clinical trials in America and Europe have shown that Riluzole leads to a prolongation of tracheotomy-free survival of about 15% in ALS patients as compared to those taking placebo; on average, this is about a 3 month improvement in survival overall. Rilutek® is a glutamate release inhibitor that is thought to decrease glutamate mediated motor neuron cell death in ALS patients. Rilutek® is generally well tolerated among patients but does not treat the symptoms nor delay the onset of the disease. Other treatments currently in clinical trials include the cycloxygenase inhibitor celecoxib (Celebrex®), minocycline, and the compound TCH-346. Clearly, it would be of great importance to have any therapy that slowed this process, even if it only benefited the subset of ALS patients with SOD1 mutations.

### SUMMARY OF THE INVENTION

The findings described above suggest that an important approach to ameliorating or preventing ALS is to inactivate expression of SOD1 protein. It is for this reason that we have devised the present assay. Our hypothesis is that any therapies that reduce expression of the SOD1 protein by the SOD1 gene will be beneficial because they will reduce levels of the SOD1 protein.

The invention provides a novel platform for drug screening in ALS. The assay has been developed in a high through-put format to allow rapid screening for compounds and small molecules that reduce the activity of the human SOD1 promoter. The format of this assay allows for the rapid screening of large libraries of compounds in an automated setting. The assay is cell based, and therefore provides a reasonable representation of the in vivo environment increasing the probability that hit compounds will show efficacy in animal models.

An important aspect of the assay is that there is no reliance on a specific disease mechanism. The assay focuses on the reduction in SOD1 mRNA transcript and protein levels as this is expected to provide therapeutic benefits in SOD1-mediated ALS.

In one aspect of the invention, a method for detecting an inhibitor of SOD1 transcription is provided. In one embodiment of the invention, a cell containing an expression vector with a SOD1 promoter sequence operably linked to a nucleotide sequence which encodes a detectable marker is contacted with a candidate inhibitor molecule. The cell is incubated for a time sufficient for expression of a detectable marker.

In another embodiment of the invention, the SOD1 promoter sequence is SEQ ID NO:1. In a further embodiment of the invention, the SOD1 promoter sequence is SEQ ID NO:5. In a further embodiment the nucleotide encoding sequence that encodes a detectable marker is selected from the group consisting of fluorescence markers, epitope tags, and enzyme tags. In another embodiment the detectable marker is green fluorescent protein (GFP) or enhanced green fluorescent protein (EGFP). In a further embodiment the detectable marker is luciferase.

In one embodiment of the invention, the expression vector further includes a multiple cloning site downstream of the promoter sequence for cloning a nucleotide sequence that encodes a protein.

In another embodiment of the invention the cell is a mammalian cell. In a preferred embodiment the cell is a PC12 cell.

In an embodiment of the invention the detectable marker is detected as a signal. In a second embodiment the signal detected is fluorescence of green fluorescent protein (GFP) or enhanced green fluorescent protein (EGFP). In another embodiment the signal detected is luminescence of a luciferase substrate. In a further embodiment a decrease in green fluorescent protein (GFP) fluorescence indicates that the candidate inhibitor molecule inhibits SOD1 transcription. In another embodiment a decrease in the enzyme tag indicates that the candidate inhibitor molecule inhibits SOD1 transcription. In yet another embodiment the cell and inhibitor molecule are incubated in a multiwell plate. In still a further embodiment the cell is contacted with a plurality of inhibitor molecules.

In another aspect of the invention, a method for preparing an assay system is provided. In one embodiment a nucleotide sequence that encodes a protein is cloned into an expression vector which includes a SOD1 promoter sequence operably linked to the nucleotide encoding sequence. Preferred SOD1 promoter sequences include SEQ ID NOs:1 and 5. In a second embodiment the nucleotide encoding sequence encodes green fluorescent protein (GFP) or enhanced green fluorescent protein (EGFP) or luciferase. In a further embodiment a multiple cloning site downstream of the promoter sequence for cloning a nucleotide sequence that encodes a protein is included. In another embodiment the vector is transferred into an expression system.

In an embodiment of the invention, the expression vector is transfected or transformed into a cell. In a preferred embodiment the cell is a mammalian cell. More preferably, the cell is a PC12 cell. In still a further embodiment the expression vector is a cell-free transcription-translation system.

### BRIEF DESCRIPTION OF THE DRAWINGS

FIG. 1 depicts a SOD1 promoter-EGFP construct.

FIG. 2 is a graph showing the relative expression levels after treatment of transfected PC12 cells with increasing mitomycin C (MC) concentrations (n=16; UT=untreated).

FIG. 3 depicts the results of Western blot analysis showing non-specific repressor of SOD1 promoter activity. The membrane was probed initially with a sheep anti-SOD1 antibody (Calbiochem), then stripped and reprobed with a mouse anti-actin antibody (Sigma).

FIG. 4 depicts a SOD1 promoter-pGL3 construct (SEQ ID NO:5).

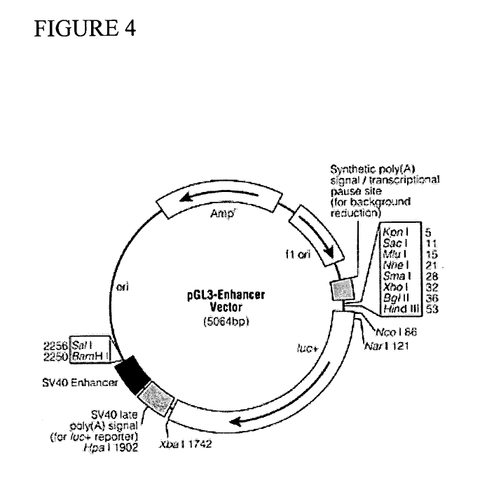

### DESCRIPTION OF SEQUENCES

SEQ ID NO:1—exemplary nucleotide sequence of a human SOD1 promoter variant with 50 bp deletion relative to other SOD1 promoter sequences.

SEQ ID NO:2—nucleotide sequence of pEGFP-1 vector.

SEQ ID NO:3—oligonucleotide used in PCR (forward primer) containing Xho I restriction site.

SEQ ID NO:4—oligonucleotide used in PCR (reverse primer) containing Hind III restriction site.

SEQ ID NO:5—nucleotide sequence of a second human SOD1 promoter sequence.

SEQ ID NO:6—nucleotide sequence of pGL3 enhancer vector.

### DETAILED DESCRIPTION OF THE INVENTION

Amyotrophic lateral sclerosis (ALS) is a progressive neurodegenerative condition with a prevalence of 2-3 per 100,000 people, involving the loss of large motor neurons in the brain and spinal cord. The incidence of ALS is 1 per 100,000 people with approximately 5,000 new cases diagnosed annually. The age of symptom onset is between the ages of 40 and 70 with men more often affected than women. ALS is characterized by progressive weakness, atrophy and spasticity, leading to paralysis, respiratory failure and death within 5 years of onset.

Familial ALS accounts for 10% of all ALS cases and approximately 25% of these cases are due to mutations in the Cu/Zn superoxide dismutase gene (SOD1) (Gurney, M. E., et al., (1996), Benefit of vitamin E, riluzole, and gabapentin in a transgenic model of familial amyotrophic lateral sclerosis, Ann Neurol, 39:147-57). The process of motor neuron degeneration in mutant SOD1 patients and in the transgenic mutant SOD1 ALS mice is dependent on the expression of mutant SOD1 protein, although the basis for the cytotoxicity of the mutant SOD1 protein is not well defined. We believe that the assay of the present invention will allow high through-put testing for compounds that can reduce expression of the SOD1 gene. This has immediate therapeutic implications for patients with ALS derived from mutant SOD1. It is conceivable that, in the long term, this assay may also have implications for other types of ALS.

The present invention provides a method of identifying inhibitors of SOD1 expression. The invention provides an expression vector, comprising a SOD1 promoter (e.g. SEQ ID NO:1 or SEQ ID NO:5), operably linked to a nucleotide coding sequence encoding a detectable marker. In some preferred embodiments, the nucleotide sequence encodes green fluorescent protein (GFP), enhanced green fluorescent protein (EGFP) or luciferase. The expression vector is contacted with a candidate inhibitor molecule, and the expression of the polypeptide encoding sequence is detected. Polypeptide or nucleic acid expression products can be detected. In a further aspect the method is a cell-based, high through-put screening assay with fluorescence-based detection.

As used herein, a “vector” may be any of a number of nucleic acids into which a desired sequence may be inserted by restriction and ligation for transport between different genetic environments or for expression in a host cell. The vectors of the invention preferably are composed of DNA, although RNA vectors are alternatives. Vectors include, but are not limited to, plasmids, phagemids, bacteria and virus genomes, such as adenovirus and poxvirus.

An expression vector is one into which a desired DNA sequence may be inserted by restriction and ligation such that it is operably joined to regulatory sequences and may be expressed as an RNA transcript. The expression vector is one which is able to replicate in a host cell or be replicated after its integration into the genome of a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase.

Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein).

A selection marker should be such that it allows—i.e. under appropriate selection conditions—host cells and/or host organisms that have been (successfully) transformed with the nucleotide sequence of the invention to be distinguished from host cells/organisms that have not been (successfully) transformed. Some preferred but non-limiting examples of such markers are genes that provide resistance against antibiotics (such as kanamycin or ampicillin), genes that provide for temperature resistance, or genes that allow the host cell or host organism to be maintained in the absence of certain factors, compounds and/or (food) components in the medium that are essential for survival of the non-transformed cells or organisms, (such as G418 resistance).

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region is operably joined to a coding sequence if the promoter region is capable of effecting transcription of that DNA sequence such that the resulting transcript can be translated into the desired protein or polypeptide. Such a promoter may be a constitutive promoter or an inducible promoter, and may also be such that it (only) provides for expression in a specific stage of development of the host cell or host organism, and/or such that it (only) provides for expression in a specific cell, tissue, organ or part of a multicellular host organism.

A polypeptide-encoding nucleotide sequence may include the entire coding sequence of a protein, or regions of a coding sequence that encode fragments of a protein. Regions of a protein includes but is not limited to domains, such as the N-domain or the C-domain, and subunits of proteins. In one embodiment, a nucleotide sequence encoding a small interfering RNA molecule (siRNA) or other detectable RNA is inserted in place of the polypeptide-encoding nucleotide sequence.

In accordance with the invention, a polypeptide-encoding nucleotide sequence is placed under the control of the human superoxide dismutase (SOD1) promoter sequence. The invention also provides that a fragment of the SOD1 promoter may be used. The fragment may be of any integer in the range of 29 to 3678 base pairs (Kim H T, et al., Study of 5′-flanking region of human Cu/Zn superoxide dismutase, Biochem Biophys Res Commun. (1994), 201(3):1526-33; Seo S J, et al., Sp1 and C/EBP-related factor regulate the transcription of human Cu/Zn SOD gene, Gene, (1996), 178(1-2):177-85; Minc E, et al., The human copper-zinc superoxide dismutase gene (SOD1) proximal promoter is regulated by Sp1, Egr-1, and WT1 via non-canonical binding sites, J Biol Chem., (1999), 274(1):503-9).

The promoters of the invention include a long SOD1 promoter (SEQ ID NO:5) and a shorter SOD1 promoter (SEQ ID NO:1). The shorter SOD1 promoter contains a 50 base pair deletion of residues 473 to 522 of the long SOD1 promoter sequence. This deletion is a common polymorphic variation present in many ALS patients.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Especially, such 5′ non-transcribed regulatory sequences will include a SOD1 promoter region which includes a promoter sequence for transcriptional control of the operably joined gene. Regulatory sequences may also include enhancer sequences or upstream activator sequences as desired.

The vectors of the invention may optionally include 5′ leader or signal sequences. A leader sequence should be such that—in the intended host cell or host organism—it allows for the desired post-translational modifications and/or such that it directs the transcribed mRNA to a desired part or organelle of a cell. A leader sequence may also allow for secretion of the expression product from said cell. As such, the leader sequence may be any pro-, pre-, or prepro-sequence operable in the host cell or host organism. In some instances, the nucleotide sequences inserted into the expression vector of the invention may include sequences that encode signal peptides. The signal sequence may refer to a region coding for a portion of a protein that is later cleaved off. The invention embraces each of these sequences with, or without, the portion of the sequence that encodes a signal peptide.

For some further non-limiting examples of the selection markers, leader sequences, expression markers and further elements that may be present/used in the genetic constructs of the invention—such as terminators, transcriptional and/or translational enhancers and/or integration factors—reference is made to the general handbooks such as J. Sambrook, et al., eds, Molecular Cloning: A Laboratory Manual, Second Edition, Cold Spring Harbor Laboratory Press, Cold Spring Harbor, N.Y., 1989, F. M. Ausubel, et al., eds, Current Protocols in Molecular Biology, John Wiley & Sons, Inc., New York, W. B. Wood et al., “The nematode Caenorhabditis elegans”, Cold Spring Harbor Laboratory Press (1988) and D. L. Riddle et al., “C. ELEGANS II”, Cold Spring Harbor Laboratory Press (1997), as well as to the examples that are given in WO 95/07463, WO 96/23810, WO 95/07463, WO 95/21191, WO 97/11094, WO 97/42320, WO 98/06737, WO 98/21355, U.S. Pat. No. 6,207,410, U.S. Pat. No. 5,693,492 and EP 1 085 089. Other examples will be clear to the skilled person.

Endonuclease restriction sites are regions at which the vector may be cut in a determinable fashion and into which a desired DNA sequence may be ligated such that the new recombinant vector retains its ability to replicate in the host cell. A multiple cloning site, consisting of many endonuclease restriction sites, is included in the vector to allow insertion of a nucleotide sequence. The multiple cloning site is located downstream of a nucleotide sequence that includes a SOD1 promoter, allowing the insertion of a nucleotide sequence encoding a detectable marker, e.g. polypeptide or nucleic acid. Multiple cloning sites may include, but are not limited to, restriction sites for endonucleases such as BspE I, Sal I, BamH I, Bsm I, Acc I, Xma I, Sma I, Spe I, Sph I, Kpn I, Acc65 I, Ahd I, Cla I, BspD I, Not I, Eag I, Fse I, NgoM I, Nae I, BseR I, Pme I, Pml I, Aat I, EcoR I, BstE II, BstB I, Bsu36I, Age I, Hind III, Hpa I, Bgl I, Bgl II, Mlu I, Rsr I, Nco I, Nde I, Sca I, Sac I, Sac II, Apa I, Bsp120 I, Xma I, Eco47 III, Ecl136 II, Pst I, Asp718 I, Swa I, SnaB I, Xba I, and Xho I.

Another aspect of the invention provides a method of screening for inhibitors using a stable cell line or transiently transfected cells. The expression vector may be transformed or transfected into a cell to create such a stable cell line or to create a transiently transfected cell.

Various techniques may be employed for introducing the vector of the invention containing a nucleic acid molecule of interest into a cell. The term “transfection” as used herein refers to the introduction of foreign DNA into cells. Such techniques include calcium phosphate precipitate transfection, DEAE transfection, transfection or infection with viruses, liposome-mediated transfection (lipofection), ballistic transformation, (micro-)injection, transfection, electroporation and the like. Stable transfection of a cell line is preferred, and this can be tested using selectable markers present in the vector. The presence of a selectable marker allows the selection of those cells which contain the vector. Selectable markers include but are not limited to for example, antibiotic resistance genes and mitomycin resistance genes. Selectable markers are well known to those of ordinary skill in the art.

For certain uses, it is preferred to target the vector containing a nucleic acid molecule to particular cells. In such instances, a vehicle used for delivering a nucleic acid molecule of the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the vector containing a nucleic acid molecule delivery vehicle. Especially preferred are monoclonal antibodies.

Liposomes are commercially available from Life Technologies, Inc., for example, as LIPOFECTIN™ and LIPOFECTACE™, which are formed of cationic lipids such as N-[1-(2,3 dioleyloxy)-propyl]-N,N,N-trimethylammonium chloride (DOTMA) and dimethyl dioctadecylammonium bromide (DDAB). Methods for making liposomes are well known in the art and have been described in many publications. Liposomes also have been reviewed by Gregoriadis, G. in Trends in Biotechnology, 3:235-241 (1985). Where liposomes are employed to deliver the vector of the invention containing a nucleic acid molecule, proteins that bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acid molecules into cells, as is known by those skilled in the art.

The vector may also be added to a cell free transcription-translation system. These systems include but are not limited to the Expressway™ Salmonella typhimurium SP6, Escherichia coli T3 and E. coli T7 phage systems (Invitrogen), and the TNT® Salmonella typhimurium SP6, E. coli T3 or E. coli T7 coupled reticulocyte lysate system (Promega), and the TNT® Salmonella typhimurium SP6, E. coli T3, or E. coli T7 coupled wheat germ extract system (Promega).

The host cells and cell lines, may be prokaryotic (e.g., E. coli), or eukaryotic (e.g., PC12 cells, CHO cells, COS cells, BHK-cells, HeLa cells, HEPG2 cells, 3T3L1 adipocytes, CTC12 cells and L6 myotubes, yeast expression systems and recombinant baculovirus expression in insect cells). Especially useful are mammalian cells such as human, mouse, rat, hamster, pig, goat, primate, etc. The host cell is preferably a cell or cell line derived from a human. In one method the cell line is rat PC12 pheochromocytoma.

To produce/obtain expression of the polypeptide-encoding nucleotide sequence, the transformed host cell or transformed host organism may generally be kept, maintained and/or cultured under conditions such that the (desired) polypeptide-encoding nucleotide sequence is expressed/produced by the vector of the invention. Suitable conditions will be clear to the skilled person and will usually depend upon the host cell used, as well as on the regulatory elements of the vector of the invention that control the expression of the (relevant) polypeptide-encoding nucleotide sequence.

Generally, suitable conditions may include the use of a suitable medium, the presence of a suitable source of food and/or suitable nutrients, the use of a suitable temperature, and optionally the presence of a suitable inducing factor or compound (e.g. when the vector of the invention has polypeptide-encoding nucleotide sequences that are under the control of an inducible promoter); all of which may be selected by the skilled person. Again, under such conditions, the polypeptide-encoding nucleotide sequences may be expressed in a constitutive manner, in a transient manner, or only when suitably induced.

It will also be clear to the skilled person that the polypeptide encoded by the vector of the invention may (first) be generated in an immature form, which may then be subjected to post-translational modification, depending on the host cell used.

The invention provides efficient methods of identifying pharmacological molecules or lead compounds for molecules that reduce the transcription of SOD1. Generally, the screening methods involve assaying for compounds which modulate (up- or down-regulate) the level of expression of a detectable marker as an indicator of SOD1 transcription. It is understood that any mechanism of action described herein for the transcription-reduction molecules is not intended to be limiting, and the scope of the invention is not bound by any such mechanistic descriptions provided herein.

As noted above, the invention relates in some aspects to the identification and testing of candidate inhibitor molecules that can reduce the transcription of SOD1. In some aspects of the invention, the transcription-reduction molecules are isolated nucleic acid molecules, that are useful for practicing the invention. In other embodiments, the compositions include isolated polypeptides, that are encoded by the above-described nucleic acid molecules. The polypeptides used in the methods of the invention embrace polypeptides as well as polypeptide fragments. The transcription-reduction polypeptides of the invention include fragments, (i.e. pieces) of transcription-reduction molecules. These fragments are shorter than the full-length transcription-reduction molecules. The transcription-reduction polypeptides and fragments of the invention can be screened for reducing transcription using the same type of assays as described herein (e.g. in the Examples section). Using such assays, the transcription-reduction molecules that have the best inhibitory activity can be identified.

As used herein, the term “inhibitor molecule” or “transcription-reduction molecule” means a molecule that inhibits or reduces the normal (control) level of transcription of SOD1. The transcription-reduction molecules of the invention may include small molecules, chemicals, polypeptides (for example, competitive ligands and antibodies, or antigen-binding fragments thereof), and may also include nucleic acids. The transcription-reduction molecules are identified using the assays provided herein, including those in the Examples section. For example, a transcription-reduction molecule may be tested for its ability to reduce SOD1 transcription. To test the ability of a transcription-reduction molecule to reduce the transcription of SOD1, a cell which includes an expression vector comprising a SOD1 promoter and a detectable marker is contacted with a candidate transcription-reduction molecule and the level of expression of the detectable marker is compared to the level of expression of the detectable marker in the absence of a candidate transcription-reduction molecule.

A candidate inhibitor molecule that produces a decrease in the level of GFP, or other detectable marker, is considered an inhibitor of SOD1 transcription. The decrease in the detectable marker is at least about 5%, 10%, 15%, 20%, 25%, 30%, 35%, 40%, 45%, 50%, 55%, 60%, 65%, 70%, 75%, 80%, 85%, 90%, 95% or 99%. In preferred embodiments, a decrease of about 35% or more is detected. In particularly preferred embodiments, a decrease in fluorescence of about 50% or more is detected.

The transcription-reduction molecules of the invention may also include nucleic acids that encode, molecules that reduce transcription and fragments thereof, nucleic acids that bind to other nucleic acids, (e.g. for antisense or RNAi methods), or may be polypeptides that reduce the transcription of SOD1. Such polypeptides include, but are not limited to, antibodies or antigen-binding fragments thereof.

Candidate inhibitor molecules useful in accordance with the invention encompass numerous chemical classes, although typically they are organic compounds. Preferably, the candidate inhibitor molecules are small organic compounds, i.e., those having a molecular weight of more than 50 Daltons (Da.) yet less than about 2500 Da., preferably less than about 1000 Da. and, more preferably, less than about 500 Da. Candidate inhibitor molecules comprise functional chemical groups necessary for structural interactions with proteins and/or nucleic acid molecules, and typically include at least an amine, carbonyl, hydroxyl or carboxyl group, preferably at least two of the functional chemical groups and more preferably at least three of the functional chemical groups. The candidate inhibitor molecules can comprise cyclic carbon or heterocyclic structure and/or aromatic or polyaromatic structures substituted with one or more of the above-identified functional groups. Candidate inhibitor molecules also can be biomolecules such as peptides, saccharides, fatty acids, sterols, isoprenoids, purines, pyrimidines, derivatives or structural analogs of the above, or combinations thereof and the like. Where the inhibitor molecules is a nucleic acid molecule, the molecule typically is a DNA or RNA molecule, although modified nucleic acid molecules as defined herein are also contemplated.

Candidate inhibitor molecules are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily modified through conventional chemical, physical, and biochemical means. Further, known pharmacological inhibitor molecules may be subjected to directed or random chemical modifications such as acylation, alkylation, esterification, amidification, etc. to produce structural analogs of the inhibitor molecules. Examples of libraries are The ChemBridge Corporation Library (ChemBridge Corporation, San Diego, Calif.), the Natural Product Library (TimTec, Inc., Newark, Del. ) and The Prestwick Chemical Library (Prestwick Chemical, Inc., Washington, DC).

The ChemBridge Corp. library (30,000 compounds) consists of Diverse and CNS sets. The Diverse set contains compounds that were selected based on 3D pharmacophore analysis to cover the broadest part of biologically relevant pharmacophore diversity space. The CNS set is a therapeutic area focused collection of pre-designed, drug-like, small molecule compounds. Computational methods are applied to select compounds with increased probability of oral bioavailability and blood brain barrier penetration. The natural product library contains 2000 natural/semi-natural products with a broad diversity of acceptable chemical structures purified to a 95% screening grade or chemically re-synthesized. Compounds that may be problematic to a cell based assay system, e.g. very high molecular weight species and simple sugars are filtered out. The Prestwick Chemical Library is a collection of 1120 high-purity chemical compounds (all off patent) carefully selected for: structural diversity, broad spectrum covering several therapeutic areas (from neuropsychiatry to cardiology, immunology, anti-inflammatory, analgesia and more) and known safety and bioavailability in humans.

The invention provides a method of screening for inhibitor molecules using high through-put screening. A high through-put screening assay is an assay that allows the screening of one or more inhibitor molecule. Generally the screening method involves assaying for inhibitor molecules that reduce transcription controlled by the SOD1 promoter. The assay mixture comprises at least one candidate inhibitor molecule. Typically, a plurality of assay mixtures are run in parallel with different inhibitor molecule concentrations to obtain a different response to the various concentrations. Typically, one of these concentrations serves as a negative control, i.e., at zero concentration of candidate inhibitor molecule or at a concentration of candidate inhibitor molecule below the limits of assay detection. The high through-put screening assay may be based in a multiwell plate. In one aspect, a cell is contacted with at least one candidate inhibitor molecule in a multiwell plate. The level of transcription is determined using a detectable marker, for example green fluorescent protein (GFP), operably linked to the SOD1 promoter.

A variety of other reagents also can be included in the mixture. These include reagents such as salts, buffers, neutral proteins (e.g., albumin), detergents, etc. which may be used to facilitate optimal protein-protein and/or protein-nucleic acid binding. Such a reagent may also reduce non-specific or background interactions of the reaction components. Other reagents that improve the efficiency of the assay such as protease inhibitors, nuclease inhibitors, antimicrobial agents, and the like may also be used.

An exemplary transcription assay is described herein, which may be used to identify candidate inhibitor molecules that modulate the transcription of SOD1. In general, the mixture of the foregoing assay materials is incubated under conditions whereby, in the presence of the candidate inhibitor molecules, the transcription of SOD1 is reduced, although in some embodiments the candidate inhibitor molecule may be one that increases the transcription of SOD1. The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 0.1 and 72 hours. After incubation, the transcription of SOD1 is detected by any convenient method available to the user. It is contemplated that cell-based assays as described herein can be performed using cell samples and/or cultured cells. Biopsy cells and tissues as well as cell lines grown in culture are useful in the methods of the invention.

A variety of methods may be used to detect the expression of a polypeptide-encoding sequence. Detection may be effected in any convenient way for cell-based assays such as use of a detectable marker. For cell based and cell-free assays, one of the components usually comprises, or is coupled to, a detectable marker. In one method, expression is detected using the expression of a detectable marker. In a preferred method the detectable marker is green fluorescent protein (GFP) or EGFP. Other markers may be used to detect the expression level of a polypeptide-encoding sequence, for example the Luciferase reporter system, and the chloramphenicol acetyl transferase (CAT) gene. The method of detection of the marker may depend on the nature of the marker and other assay components. A wide variety of markers can be used, for example, the marker may be directly detected through optical or electron density, radioactive emissions, nonradiative energy transfers, luminescence, fluorescence for example green fluorescent protein (GFP), etc. or indirectly detected with antibody conjugates, strepavidin-biotin conjugates, epitope tags such as the FLAG epitope, enzyme tags such as horse-radish peroxidase, etc. Methods for detecting and using markers are well known in the art.

As will be understood by the person of ordinary skill in the art, it is desirable to measure the amount of SOD1 transcription reduction in a linear range of the assay system, such that small but significant decreases in detectable marker (e.g. fluorescence) relative to control well (e.g., devoid of a candidate inhibitor molecule) may be observed. It is well within the skill of the art to determine a volume and concentration of a candidate inhibitor molecule which causes a suitable response in cells so that SOD1 transcription reduction may be reliably detected.

At a suitable time after addition of the candidate inhibitor molecule to reduce SOD1 transcription, the plate is moved, if necessary, so that the cell-containing assay well is positioned for measurement of fluorescence emission or other detectable marker properties. Because a change in the fluorescence or other signal may begin within the first few seconds after addition of candidate inhibitor molecules, it is sometimes desirable to align the assay well with the fluorescence reading device as quickly as possible, with times of about two seconds or less being desirable. Depending on the detectable marker used in the method, fluorescence is measured at about 1, 2, 3, 4, 5, 10, 15, 20, 24, 36, 48, 60, or 72 hours after the addition of candidate inhibitor molecules. In a preferred embodiment of the invention, fluorescence is read after 72 hours incubation. One of ordinary skill in the art is able to determine a suitable incubation time for reading fluorescence or other detectable signal. In preferred embodiments of the invention, where the apparatus is configured for detection through the bottom of the well(s) and inhibitor molecules are added from above the well(s), fluorescence readings may be taken substantially continuously, since the plate does not need to be moved for addition of candidate inhibitor molecule or other reagent. The well and detector (e.g. a fluorescence-reading device) should remain aligned for a predetermined period of time suitable to measure and record the change in signal, e.g. GFP or EGFP fluorescence. In preferred embodiments of the invention the fluorescence after activation is read and recorded until the fluorescence change is maximal and then begins to reduce. An empirically determined time period may be chosen which covers the transient rise and fall (or fall and rise) of SOD1 transcription levels in response to addition of the candidate inhibitor molecule. When the apparatus is configured to detect fluorescence from above the plate, it is preferred that the sides of the wells are colored black to reduce the background fluorescence and thereby decrease the noise level in the fluorescence reader.

After finishing reading and recording the fluorescence in one well or set of wells, the just described apparatus steps are repeated with the next well or set of wells in the series so as to measure pre-reagent fluorescence, add candidate inhibitor molecule and measure and record the transient change, if any, in fluorescence. The apparatus of the present invention preferably is programmable to begin the steps of an assay sequence in a predetermined first well (or row or column of wells) and proceed sequentially down the columns and across the rows of the plate in a predetermined route through well number n.

In assays of cells treated with candidate inhibitor molecules to cause a decrease in SOD1 transcription, it is preferred that the data (e.g. fluorescence) from replicate wells of cells treated with the same compound are collected and recorded (e.g., stored in the memory of a computer) for calculation of fluorescence and/or SOD1 transcription.

In assays of candidate inhibitor molecules that reduce SOD1 transcription, the results can be expressed as a percentage of the maximal decrease caused by candidate inhibitor molecules. The maximal signal (e.g. fluorescence) decrease caused by an inhibitor molecule is defined as being 100% response. For inhibitor molecules effective for reducing SOD1 transcription, the maximal fluorescence recorded after addition of a candidate inhibitor molecule to a well is detectably lower than the fluorescence recorded in the absence of an inhibitor molecule. The detectable signal (e.g. fluorescence indicator) based assays of the present invention are thus useful for rapidly screening candidate inhibitor molecules to identify those that reduce SOD1 transcription that ultimately results in reduced SOD1 protein expression.

In another of its aspects the invention entails automated assays. Automation of the assays of the invention can be performed as described in U.S. Pat. No. 6,057,114. Automated assays, including drug screening assays, may be carried out by incubating the cells (e.g., PC12 cells) that include a SOD1 promoter-containing vector with candidate inhibitor molecule(s) to reduce SOD1 transcription, for an amount of time sufficient for the candidate inhibitor molecule(s) to reduce SOD1 transcription, and measuring the level of a detectable marker (e.g. fluorescence of GFP) in the cells as compared to the level of fluorescence in either the same cell, or substantially identical cell, in the absence of the candidate inhibitor molecule. Automation can provide increased efficiency in conducting the assays and increased reliability of the results by permitting multiple measurements over time.

For example, to accomplish rapid candidate inhibitor molecule addition and rapid reading of a fluorescence response, a fluorometer can be modified by fitting an automatic pipetter and developing a software program to accomplish precise computer control over both the fluorometer and the automatic pipetter. By integrating the combination of the fluorometer and the automatic pipetter and using a microcomputer to control the commands to the fluorometer and automatic pipetter, the delay time between reagent addition and fluorescence reading can be significantly reduced. Moreover, both greater reproducibility and higher signal-to-noise ratios can be achieved as compared to manual addition of reagent because the computer repeats the process precisely time after time. Moreover, this arrangement permits a plurality of assays to be conducted concurrently without operator intervention. Thus, with automatic delivery of reagent followed by multiple fluorescence measurements, reliability of the fluorescent dye-based assays as well as the number of assays that can be performed per day are advantageously increased.

### EXAMPLES

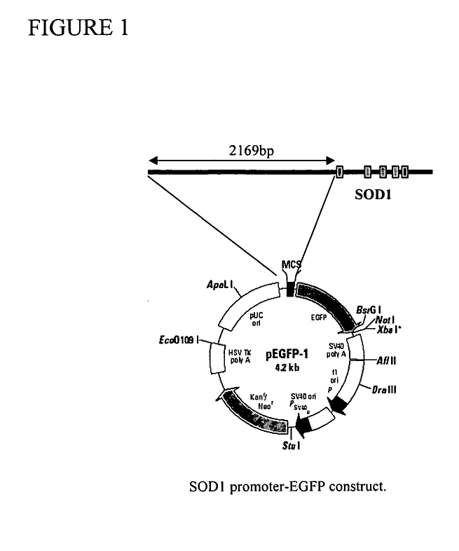

We have developed a cell-based, fluorescent high through-put screening assay to identify compounds that reduce SOD1 expression using a stable rat pheochromocytoma PC12 cell line with 2.2 kb of human SOD1 promoter sequence driving EGFP expression (FIG. 1).

The human SOD1 promoter was amplified by PCR from a control DNA sample using primers 5′ AGGCTCGAGAGAATCACTTGAACCCAGCA 3′ (SEQ ID NO:3) and 5′ CGTAAGCTTCGCCATAACTCGCTAGGCCACGC 3′ (SEQ ID NO:4). Restriction sites were incorporated into the primers although these were not used for the cloning but to aid transfer into additional vectors in the future. PCR reactions were carried out in a final volume of 40 μl on a thermocycler (Hybaid). PCR reactions contained a final concentration of 3 U Taq polymerase (New England Biolabs, Beverly, Mass.), 150 ng of DNA, 1×PCR buffer, 0.5 nM of each dNTP, 40 pmoles of forward and reverse primers (MWG-Biotech, High Point, N.C.), 2.5 mM MgCl₂, and 5% DMSO. PCR conditions used were an initial 10 min denaturation at 95° C., 33 cycles of 1 min at 95° C., 1 min annealing at 65° C., 2.5 min extension at 72° C. and a final extension for 20 mins at 72° C. The PCR product was ligated into the pGEM-T Easy vector (Promega, Madison, Wis.); the insert was then cut out using restriction endonucleases SalI and SacII and ligated into the pEGFP-1 vector (Clontech, Palo Alto, Calif.) cut with SalI and SacII.

PC12 cells were chosen for this assay because they demonstrate some neuronal characteristics, can be differentiated after the addition of nerve growth factor and have high transfection efficiency. The pEGFP plasmid system (Clontech, Palo Alto, Calif.) was chosen since this red-shifted variant of GFP allows brighter fluorescence and higher expression in mammalian cells than standard GFP (Delagrave, S., et al., (1995), Red-shifted excitation mutants of the green fluorescent protein, Biotechnology (NY), 13:151-4.).

PC12 cells were stably transfected with the SOD1 promoter construct using Lipofectamine 2000 (Invitrogen, Carlsbad, Calif.). Clonal cell lines were selected based on high levels of EGFP expression. PC12 stable cell lines were cultured in DMEM-F12 (Gibco, Carlsbad, Calif.) containing 10% (v/v) horse serum, 5% (v/v) FBS, 1×penicillin, 1×streptomycin and 500 μg/ml G418.

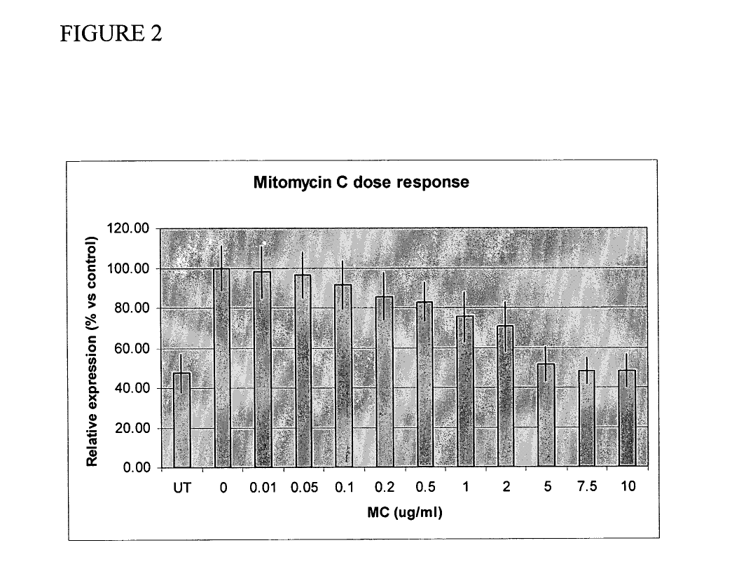

Initial testing of the assay has been carried out using a known inhibitor of SOD1 expression, mitomycin C (Cho C., et al., (1997), The transcriptional repression of the human Cu/Zn superoxide dismutase(sod1) gene by the anticancer drug, mitomycin C(MMC), Biochem Mol Biol Int., 42:949-56). The high through-put screening format involved plating 50,000 cells/well with compounds (1 to 25 μM) in a 96-well plate and incubation for 72 hrs. The cells were washed three times with PBS using an automatic plate washer and lysed (RIPA buffer −50 mM Tris-HCl (pH 7.4), 1% NP-40, 0.25% Na-deoxycholate, 150 mM NaCl, 1 mM EDTA, 1 mM PMSF, 1 mM Na₃VO₄, 1 mM NaF and Aprotinin, leupeptin, pepstatin (1 microgram/ml each)). The level of EGFP fluorescence was read on a Wallac plate reader (Perkin Elmer, Boston, Mass.) (excitation 485 nm, emission 535 nm). After treatment of the PC12 stable cell line for 24 hrs with increasing mitomycin C concentration a progressive decrease in fluorescence was noted (FIG. 2).

A mixed library of 21,000 compounds was screened. The compounds were obtained from The Chembridge Corporation Library (ChemBridge Corporation, San Diego, Calif.), The Natural Product Library (TimTec, Inc., Newark, Del.) and The Prestwick Chemical Library (Prestwick Chemical, Inc., Washington, DC). For the high throughput screening the compounds are preplated in 96-well black wall/clear bottom plates (Perkin-Elmer, Boston, Mass. cat#1450-573) at a concentration of 1 μM and 100 μl of media added per well. The stably transfected PC12 cells expressing the SOD1 promoter-EGFP reporter construct were plated at 50,000 cells/well using an automated cell plater and incubated for 72 hrs.

After incubation the plates were washed three times with 150 μl of 1×PBS using an automatic plate washer and lysed with 150 μl of RIPA buffer (150 mM NaCl, 1% Triton X, 0.5% Sodium deoxycholate, 0.5% SDS, 40 mM Tris-HCl (pH8)). The level of EGFP fluorescence was then read on a Wallac plate reader (Perkin-Elmer, Boston, Mass.) (excitation 485 nm, emission 535 nm). Compounds that reduce EGFP fluorescence more than 50% compared to untreated cells were designated as hits from the primary assay. 41 hits were obtained from the screening of the combined library of 21,000 compounds.

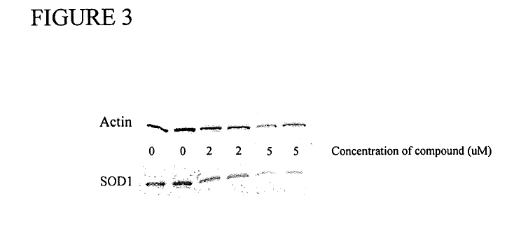

Analysis of protein expression was performed (FIG. 3). Cell lysates were analyzed using Western blot analysis. The membrane was probed initially with a sheep anti-SOD1 antibody (Calbiochem, La Jolla, Calif.), then stripped and re-probed with a mouse anti-actin antibody (Sigma, St. Louis, Mo.).

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

1. A method for detecting an inhibitor of SOD1 transcription comprising
contacting a cell comprising an expression vector with a SOD1 promoter sequence operably linked to a nucleotide sequence that encodes a detectable marker with a candidate inhibitor molecule, and
incubating the cell for a time sufficient for expression of the detectable marker,
wherein a decrease in fluorescence of a fluorescence marker indicates that the candidate inhibitor molecule inhibits SOD1 transcription.

2. The method of claim 1, wherein the SOD1 promoter sequence is SEQ ID NO:1.

3. The method of claim 1, wherein the SOD1 promoter sequence is SEQ ID NO:5.

4. The expression vector of claim 1, wherein the detectable marker is selected from the group consisting of fluorescence markers, epitope tags and enzyme tags.

5. The method of claim 4, wherein the detectable marker is green fluorescent protein or enhanced green fluorescent protein.

6. The method of claim 4, wherein the detectable marker is luciferase.

7. The method of claim 1, wherein the expression vector further includes a multiple cloning site downstream of the promoter sequence for inserting a nucleotide sequence.

8. The method of claim 1, wherein the cell is a mammalian cell.

9. The method of claim 8, wherein the cell is a PC12 cell.

10. The method of claim 1, wherein the detectable marker is detected as a signal.

11. The method of claim 10, wherein the signal detected is fluorescence of green fluorescent protein (GFP) or EGFP.

12. The method of claim 10, wherein the signal detected is luminescence of a luciferase substrate.

13. The method of claim 4, wherein a decrease in fluorescence of a fluorescence marker indicates that the candidate inhibitor molecule inhibits SOD1 transcription.

14. The method of claim 4, wherein a decrease in the enzyme tag indicates that the candidate inhibitor molecule inhibits SOD1 transcription.

15. The method of claim 1, wherein the cell and candidate inhibitor molecule are contacted and incubated in a multiwell plate.

16. The method of claim 1, wherein the cell is contacted with a plurality of candidate inhibitor molecules.

17. A method for preparing an assay system comprising
cloning a nucleotide sequence into an expression vector, wherein the expression vector comprises a SOD1 promoter sequence operably linked to the nucleotide sequence, wherein the nucleotide sequence encodes GFP, EGFP or luciferase, and wherein a multiple cloning site downstream of the promoter sequence for cloning a nucleotide sequence that encodes a protein is included and
transferring the vector into an expression system.

18. The expression vector of claim 17, wherein the expression vector is transfected or transformed into a cell.

19. The method of claim 18, wherein the cell is a mammalian cell.

20. The method of claim 18, wherein the cell is a PC12 cell.

21. The method of claim 17, wherein the expression system is a cell-free transcription-translation system.


SEQUENCE LISTING (6 sequences)

SEQ ID NO: 1 - DNA, 2164 bp, Homo sapiens
agaatcacttgaacccagcaggcggaggttgcagtgagctgagattgcaccactgcactccagcctggtgacagagtgagactccatatcaaaataaatacataaataaataaaaacagtgattcttaactgggagtgatttggcaacgtctggaattatttttggttatcccagcctggcagggagggacagggtattactggcatctagtgagtaggggctagggattctactgaacatcctacagtgtacaggacagcctccacagcaaagaactgtctggcccaaaatgtccatagtgcccacattcgatgccctgcattaggaagatataaatactcttaaatatcacagagttaaattccttacccctgttctagcagagatgatattcttgcggggggagcatcttcttggcttcaacacattcttttctccatgggagatgatgccagaagagggacacggaacaggacgaccatcctacgaaggcaccacccaggcatcattagaccgtctcaaaagaagagtaattcactgtcccaaagcagctctctcgtgtctgtgggcggatcccttggcaagtttacaatgaactgaaatctgccgaacttcctggaacccaaagaaactttagccttgggcaaaggccctttggccagcatttgcactgtttatgcaaccgtttagaatatacgaattatctggagactactaccaaatacaacaggcaaaactgcaaatatgtatacttcctagaggatgataaaaaaatgtgaattgtatttctctgatagaggatgcattagagtctgagggtctaaatagcgtaaataataaataagtaaataaatcgatagtagtgtactccaaacgaggctggaatagcttctattgttgtttcacactggacttcaattaagtctcagtattttgccatactcaatattaagtactaggctggacgtggtggctcatgtctgtaatcccagcactttgggaggccgaggtgggtagatggctggcttgagctcaggagtttgaaaccagcctgggcaacatggtaaaaccccatctgtacccaaaatacaaaaatcagccaggtgtggtggcacatgcctgtggtcccaggtacttgggaggctgaggcaggaggatggcttgaacccaggaggtggaggctgcagtgagctatgatggcgccactgcactccagcctgggtgacagagcgagaccctgtctcaaaaatcaaacaaacaaccccctcgccccggacaaaagtagtttgcactattttctcatttcacaatatgtttttgaaatatttcccttgaaaggtaagtcatatttatcattcctgttgtatggaggcatcataaattatttcaccattctaccctccttgagtgttgtggcctttaggccagacaaaaacgcaggtgatgcctagaagccaactagttgccgtttggttatctgtagggttgtggccttgccaaacaggaaaaatataaaaagaataccgaattctgccaaccaaataagaaactctatactaaggactaagaaaattgcaggggaagaaaaggtaagtcccgggattgaggtgtagcgactttctataccctcagaaaactaaaaaacaagacaaaaaaatgaaaactacaaaagcatccatcttggggcgtcccaattgctgagtaacaaatgagacgctgtggccaaactcagtcataactaatgacatttctagacaaagtgacttcagattttcaaagcgtaccctgtttacatcattttgccaatttcgcgtactgcaaccggcgggccacgcccccgtgaaaagaaggttgttttctccacatttcggggttctggacgtttcccggctgcggggcggggggagtctccggcgcacgcggccccttggccccgcccccagtcattcccggccactcgcgacccgaggctgccgcagggggcgggctgagcgcgtgcgaggcgattggtttggggccagagtgggcgaggcgcggaggtctggcctataaagtagtcgcggagacggggtgctggtttgcgtcgtagtctcctgcagcgtctggggtttccgttgcagtcctcggaaccaggacctcggcgtggcctagcgagttatggcg

SEQ ID NO: 2 - DNA, 4151 bp, Artificial sequence
  pEGFP-1 vector
tagttattactagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 3 - DNA, 29 bp, Artificial sequence
  Synthetic oligonucleotide
aggctcgagagaatcacttgaacccagca

SEQ ID NO: 4 - DNA, 32 bp, Artificial sequence
  Synthetic oligonucleotide
cgtaagcttcgccataactcgctaggccacgc

SEQ ID NO: 5 - DNA, 2221 bp, Homo sapiens
agaatcacttgaacccagcaggcggaggttgcagtgagctgagattgcaccactgcactccagcctggtgacagagtgagactccatatcaaaataaatacataaataaataaaaacagtgattcttaactgggagtgatttggcaacgtctggaattatttttggttatcccagcctggcagggagggacagggtattactggcatctagtgagtaggggctagggattctactgaacatcctacagtgtacaggacagcctccacagcaaagaactgtctggcccaaaatgtccatagtgcccacattcgatgccctgcattaggaagatataaatactcttaaatatcacagagttaaattccttacccctgttctagcagagatgatattcttgcggggggagcatcttcttggcttcaacacattcttttctccatgggagatgatgccagaagagggacagaacagggcccagtaaagcatggggcctggggccagggacccccttgttcaggtgtgacgaccatcctacgaaggcaccacccaggcatcattagaccgtctcaaaagaagagtaattcactgtcccaaagcagctctctcgtgtctgtgggcggatcccttggcaagtttacaatgaactgaaatctgccgaacttcctggaacccaaagaaactttagccttgggcaaaggccctttggccagcatttgcactgtttatgcaaccgtttagaatatacgaattatctggagactactaccaaatacaacaggcaaaactgcaaatatgtatacttcctagaggatgataaaaaaatgtgaattgtatttctctgatagaggatgcattagagtctgagggtctaaatagcgtaaataataaataagtaaataaatcgatagtagtgtactccaaacgaggctggaatagcttctattgttgtttcacactggacttcaattaagtctcagtattttgccatactcaatattaagtactaggctggacgtggtggctcatgtctgtaatcccagcactttgggaggccgaggtgggtagatggctggcttgagctcaggagtttgaaaccagcctgggcaacatggtaaaaccccatctgtacccaaaatacaaaaatcagccaggtgtggtggcacatgcctgtggtcccaggtacttgggaggctgaggcaggaggatggcttgaacccaggaggtggaggctgcagtgagctatgatggcgccactgcactccagcctgggtgacagagcgagaccctgtctcaaaaatcaaacaaacaaccccctcgccccggacaaaagtagtttgcactattttctcatttcacaatatgtttttgaaatatttcccttgaaaggtaagtcatatttatcattcctgttgtatggaggcatcataaattatttcaccattctaccctccttgagtgttgtggcctttaggccagacaaaaacgcaggtgatgcctagaagccaactagttgccgtttggttatctgtagggttgtggccttgccaaacaggaaaaatataaaaagaataccgaattctgccaaccaaataagaaactctatactaaggactaagaaaattgcaggggaagaaaaggtaagtcccgggattgaggtgtagcgactttctataccctcagaaaactaaaaaacaagacaaaaaaatgaaaactacaaaagcatccatcttggggcgtcccaattgctgagtaacaaatgagacgctgtggccaaactcagtcataactaatgacatttctagacaaagtgacttcagattttcaaagcgtaccctgtttacatcattttgccaatttcgcgtactgcaaccggcgggccacgcccccgtgaaaagaaggttgttttctccacatttcggggttctggacgtttcccggctgcggggcggggggagtctccggcgcacgcggccccttggccccgcccccagtcattcccggccactcgcgacccgaggctgccgcagggggcgggctgagcgcgtgcgaggcgattggtttggggccagagtgggcgaggcgcggaggtctggcctataaagtagtcgcggagacggggtgctggtttgcgtcgtagtctcctgcagcgtctggggtttccgttgcagtcctcggaaccaggacctcggcgtggcctagcgagttatggcgaagcttacg

SEQ ID NO: 6 - DNA, 5064 bp, Artificial sequence
  pGL3 vector
ggtaccgagctcttacgcgtgctagcccgggctcgagatctgcgatctaagtaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata